(19) United States
(12) Patent Application Publication
(10) Pub. No.: US 2023/0255616 A1
(43) Pub. Date: Aug. 17, 2023

(54) INTRA-LUMEN SUTURE KNOT DEPLOYMENT

(71) Applicant: Edwards Lifesciences Corporation, Irvine, CA (US)

(72) Inventor: John Richard Carpenter, Santa Ana, CA (US)

(21) Appl. No.: 18/166,427

(22) Filed: Feb. 8, 2023

Related U.S. Application Data
(60) Provisional application No. 63/267,890, filed on Feb. 11, 2022.

(51) Int. Cl. A61B 17/04

(52) U.S. Cl. CPC A61B 17/0401; A61B 2017/0409; A61B 2017/0445; A61B 2017/0464

(57) ABSTRACT

A delivery system comprises a needle comprising a distal tip configured to pierce a valve leaflet of a heart and a lumen configured to receive one or more suture anchors, a first suture anchor configured to be delivered via the lumen of the needle, and a pusher configured to extend through the lumen of the needle and configured to press the first suture anchor beyond the distal tip of the needle.

### CROSS REFERENCE TO RELATED APPLICATION

This application claims the benefit of U.S. Application No. 63/267,890, filed Feb. 11, 2022, the disclosure all of which is incorporated by reference.

### BACKGROUND

The disclosure herein relates to cardiac valve repairs, and more particularly to surgical suture placement in connection with minimally invasive valve repair operations.

Certain medical and other procedures involve the use of sutures or other similar devices.

### SUMMARY

Described herein are one or more methods and/or devices for delivering one or more sutures and/or suture anchors to one or more valve leaflets of a heart.

In accordance with some implementations of the present disclosure, a delivery system comprises a needle comprising a distal tip configured to pierce a valve leaflet of a heart and a lumen configured to receive one or more suture anchors, a first suture anchor configured to be delivered via the lumen of the needle, and a pusher configured to extend through the lumen of the needle and configured to press the first suture anchor beyond the distal tip of the needle.

The first suture anchor may have a coiled and tubular form while within the lumen of the needle. In some instances, the delivery system further comprises a second suture anchor stacked end-to-end with the first suture anchor within the lumen of the needle.

In some instances, the pusher is configured to contact the second suture anchor to press the first suture anchor beyond the distal tip of the needle. The suture tails of the first suture anchor are configured to extend through a lumen of the pusher.

The distal tip of the needle may extend along an inner diameter of the needle. In some instances, the first suture anchor is configured to form a knot following removal from the needle.

In some instances, the pusher is configured to press against the first suture anchor beyond the distal tip of the needle to facilitate forming the knot.

Some implementations of the present disclosure relate to a method comprising delivering a needle carrying a first suture anchor within a lumen of the needle to a proximal side of a heart valve leaflet. The needle comprises a pointed tip at a distal end of the needle. The first suture anchor comprising a coiled suture and one or more suture tails. The method further comprises puncturing the proximal side of the heart valve leaflet with the pointed tip of the needle, extending the pointed tip beyond a distal side of the heart valve leaflet, pressing the first suture anchor beyond the distal end of the needle on the distal side of the heart valve leaflet, and tensioning the one or more suture tails of the first suture anchor to form the first suture anchor into a knot.

In some instances, the method further comprises extending a pusher to press the first suture anchor out of the lumen of the needle. The method may further comprise maintaining contact between the pusher and the first suture anchor beyond the distal end of the needle.

The method may further comprise maintaining contact between the pusher and the first suture anchor while tensioning the one or more suture tails of the first suture anchor. In some instances, the pusher comprises a lumen configured to receive the one or more suture tails of the first suture anchor.

In some instances, the method further comprises, before delivering the needle to the heart valve leaflet, winding the first suture anchor onto a suture winding tool and placing the first suture anchor into the lumen of the needle at the distal end of the needle.

The method may further comprise, before placing the first suture anchor in the lumen of the needle, winding a second suture anchor onto a suture winding tool and placing the second suture anchor into the lumen of the needle at the distal end of the needle.

In accordance with one or more implementations of the present disclosure, a method comprises winding a first suture into a tubular form on a suture winding tool, placing the first suture in the tubular form into a lumen of a needle at a distal end of the needle, the distal end of the needle comprising a pointed tip, delivering the needle through a valve leaflet of a heart, pressing the first suture beyond the distal end of the needle, and pulling one or more suture tails of the first suture to cause the first suture to form a knot at a distal side of the valve leaflet.

The method may further comprise extending a pusher through the lumen of the needle to press the first suture beyond the distal end of the needle. In some instances, the method further comprises pressing the first suture against the pusher to facilitate forming the knot.

In some instances, the pusher comprises a lumen configured to receive the one or more suture tails of the first suture. The method may further comprise winding a second suture into a tubular form on the suture winding tool and placing the second suture into the lumen of the needle in an end-to-end configuration with the first suture.

For purposes of summarizing the disclosure, certain aspects, advantages and novel features have been described herein. It is to be understood that not necessarily all such advantages may be achieved in accordance with any particular instance. Thus, the disclosed instances may be carried out in a manner that achieves or optimizes one advantage or group of advantages as taught herein without necessarily achieving other advantages as may be taught or suggested herein.

### BRIEF DESCRIPTION OF THE DRAWINGS

Various instances are depicted in the accompanying drawings for illustrative purposes and should in no way be interpreted as limiting the scope of the inventions. In addition, various features of different disclosed instances can be combined to form additional instances, which are part of this disclosure. Throughout the drawings, reference numbers may be reused to indicate correspondence between reference elements. However, it should be understood that the use of similar reference numbers in connection with multiple drawings does not necessarily imply similarity between respective instances associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some instances or configurations.

FIG. 1 illustrates an example representation of a heart having various features relevant to certain aspects of the present inventive disclosure.

FIG. 2 is a perspective view of a tissue anchor delivery device in accordance with one or more examples.

FIG. 3 is a cutaway view of a tissue anchor delivery device disposed at least partially within a chamber of a heart in accordance with one or more examples.

FIG. 4 shows a plurality of suture pairs passing through a cardiac tissue wall and through a pledget in accordance with one or more examples.

FIG. 5 illustrates an example suture anchor in accordance with one or more examples.

FIG. 6 illustrates a suture knot (e.g., bulky knot) formed from a suture anchor (e.g., the suture anchor of FIG. 5) in accordance with one or more instances.

FIG. 7 illustrates an overhead view of an upper/distal side of a valve (e.g., viewing the mitral valve from the left atrium) having two suture knots with a bulky knot form delivered to the upper/distal side of the valve.

FIGS. 8A and 8B illustrate portions of example needles configured for delivery of one or more suture anchors in accordance with one or more instances.

FIG. 9 illustrates another exemplary instance of a needle in accordance with one or more instances.

FIG. 10 illustrates alignment of a pusher extending through the lumen of the needle with respect to the sharp tip of the needle in accordance with one or more instances.

FIG. 11 illustrates an example delivery system for delivering one suture anchor from within a lumen of a needle in accordance with one or more instances.

FIG. 12 illustrates an example delivery system for delivering and/or deploying one or more suture anchors from within a lumen of a needle in accordance with one or more instances.

FIG. 13 (FIGS. 13-1, 13-2, 13-3, 13-4, and 13-5) is a flow diagram illustrating a process or delivering multiple leaflet anchors to a valve leaflet in accordance with one or more instances of the present disclosure.

FIG. 14 (FIGS. 14-1, 14-2, 14-3, 14-4, and 14-5) provides illustrations corresponding to steps of the process described in FIG. 13.

To further clarify various aspects of instances of the present disclosure, a more particular description of certain instances will be made by reference to various aspects of the appended drawings. It is appreciated that these drawings depict only typical instances of the present disclosure and are therefore not to be considered limiting of the scope of the disclosure. Moreover, while the figures can be drawn to scale for some instances, the figures are not necessarily drawn to scale for all instances. Instances of the present disclosure will be described and explained with additional specificity and detail through the use of the accompanying drawings.

### DETAILED DESCRIPTION

The headings provided herein are for convenience only and do not necessarily affect the scope or meaning of the claimed invention.

Although certain preferred instances and examples are disclosed below, inventive subject matter extends beyond the specifically disclosed instances to other alternative instances and/or uses and to modifications and equivalents thereof. Thus, the scope of the claims that may arise herefrom is not limited by any of the particular instances described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain instances; however, the order of description should not be construed to imply that these operations are order dependent. Additionally, the structures, systems, and/or devices described herein may be embodied as integrated components or as separate components. For purposes of comparing various instances, certain aspects and advantages of these instances are described. Not necessarily all such aspects or advantages are achieved by any particular instance. Thus, for example, various instances may be carried out in a manner that achieves or optimizes one advantage or group of advantages as taught herein without necessarily achieving other aspects or advantages as may also be taught or suggested herein.

The following description refers to the accompanying drawings, which illustrate specific instances. Other instances having different structures and operation do not depart from the scope of the disclosure.

### Overview

Instances of the present disclosure provide solutions relating to the treatment of certain structural heart conditions with multiple valve leaflet anchors and/or devices configured to deliver multiple valve leaflet anchors. Various disease processes can impair the proper functioning of one or more of the valves of the heart. These disease processes include degenerative processes (e.g., Barlow's disease, fibroelastic deficiency), inflammatory processes (e.g., rheumatic heart disease), and infectious processes (e.g., endocarditis). Additionally, damage to the ventricle from prior heart attacks (e.g., myocardial infarction secondary to coronary artery disease) or other heart diseases (e.g., cardiomyopathy) can distort the geometry of the heart causing valves in the heart to dysfunction. Many patients undergoing valve surgery, such as mitral valve surgery, suffer from a degenerative disease that causes a malfunction in a leaflet of the valve, which results in prolapse and regurgitation.

Valve regurgitation occurs when the leaflets of the valve do not close completely, thereby allowing blood to leak back into the prior chamber when the heart contracts. There are generally three mechanisms by which a valve becomes regurgitant or incompetent, including Carpentier's type I, type II and type III malfunctions. A Carpentier type I malfunction involves the dilation of the annulus such that the area of the valve orifice increases. The otherwise normally functioning leaflets do not have enough surface area to cover the enlarged orifice and fail to form a tight seal (e.g., do not coapt properly) causing regurgitation. Included in a type I mechanism malfunction are perforations of the valve leaflets, as in endocarditis. A Carpentier's type II malfunction involves prolapse of a segment of one or both leaflets above the plane of coaptation. This is the most commonly treated cause of mitral regurgitation, and is often caused by the stretching or rupturing of chordae tendineae normally connected to the leaflet. A Carpentier's type III malfunction involves restriction of the motion of one or more leaflets such that the leaflets are abnormally constrained below the level of the plane of the annulus. Leaflet restriction can be caused by rheumatic heart disease (IIIa) or dilation of the ventricle (IIIb).

Mitral valve disease is the most common valvular heart disorder, with nearly four million Americans estimated to have moderate to severe mitral valve regurgitation (“MR”), with similar numbers of individuals impacted outside of the United States. MR can result in a volume overload on the left ventricle which in turn progresses to ventricular dilation, decreased ejection performance, pulmonary hypertension, symptomatic congestive heart failure, atrial fibrillation, right ventricular dysfunction, and/or death. Successful surgical mitral valve repair can at least partially restore mitral valve competence, abolish the volume overload on the left ventricle, improve symptom status, and/or prevent adverse left ventricular remodeling. While generally safe and effective, conventional open-heart operations are invasive, result in significant disability, and require extended post-procedure recovery. Patients routinely spend five to seven days in the hospital and often are not able to return to normal daily activities for a month or more.

In many instances of mitral valve regurgitation, repair may be preferable to valve replacement. There are a variety of advantages to performing heart valve repair (e.g., mitral valve repair) using less invasive procedures while the heart is still beating, as described in detail herein. Mitral valve repair procedures may rely upon use of visualization technology, such as sonic guidance, which may have limitations that can reduce the effectiveness of such repairs. Accordingly, there is a continuing need for new procedures and devices for performing less invasive mitral valve repairs which do not require cardiac arrest and are less technologically challenging.

In some implementations, the present disclosure relates to an anchor delivery system configured to deliver multiple suture knots simultaneously, near-simultaneously, and/or during a single deployment stage. The term “suture” is used herein according to its plain and ordinary meaning and may refer to any elongate cord strip, strand, line, tie, string, ribbon, strap, or portion thereof, or other type of material used in medical procedures. One having ordinary skill in the art will understand that a wire or other similar material may be used in place of a suture. Furthermore, in some contexts herein, the terms “cord” and “suture” may be used substantially interchangeably. In addition, use of the singular form of any of the suture-related terms listed above, including the terms “suture” and “cord,” may be used to refer to a single suture/cord, or to a portion thereof. For example, where a suture knot or anchor is deployed on a distal side of a tissue portion, and where two suture portions extend from the knot/anchor on a proximal side of the tissue, either of the suture portions may be referred to as a “suture” or a “cord,” regardless of whether both portions are part of a unitary suture or cord.

While suture knots may be implanted one at a time, it may require an average of approximately seven minutes to navigate a delivery device to a valve leaflet and/or deploy a single suture knot. Accordingly, the time and/or level of invasiveness required to deploy multiple leaflets may be multiplied for each additional suture knot delivered. It is therefore advantageous to deliver and/or deploy multiple (e.g., two) suture knots during a single stage and/or by navigating a delivery device to the valve leaflet only once.

In some instances, a needle may be passed and/or slidably received through an interior lumen of a shaft and/or may be configured to carry and/or deliver at least one suture and/or suture knot to a leaflet of a heart valve. The needle may further be configured to puncture a valve leaflet and/or deploy a suture knot on a distal (e.g., atrial) side of the leaflet. The needle and/or suture knots may be deployed sequentially and/or simultaneously.

Some instances of the present disclosure relate to needles configured to carry one or more suture knots within lumens of the needles. Carrying suture knots within an interior of a needle may provide several advantages. For example, only a small portion of the needle may be extended through a valve leaflet to a distal side of the valve leaflet. Once an opening of the needle is beyond the distal side of the leaflet, the suture knots within the needle may be pushed out of the opening without requiring the needle to extend further beyond the leaflet. In this way, delivery of suture knots may be performed at various portions of a valve leaflet, including portions which are not situated below areas of significant clearance (e.g., P1 and/or P3 regions of a mitral leaflet).

As another advantage of delivering suture knots within a needle, friction between the needle and the leaflet may be reduced. For example, sutures and/or other devices carried on an exterior of a needle may result in increased friction between the delivery systems and the leaflet, which can push the leaflet upward and/or can cause tearing and/or bleeding of the leaflet. Moreover, risk of the suture knots becoming unraveled while passing through the valve leaflet can be minimized. Deployment of one or more suture knots from within a needle can require minimal force as the suture knots may be at least partially free-floating. After the suture knots are extended beyond the distal end of the needle (e.g., using a pusher), suture tails extending from the suture knots can be pulled to form the suture knots into bulky knot and/or similar forms on the distal side of the leaflet. In some instances, a suture locking mechanism may be used to lock the suture tails in place.

In some instances, one or more suture knots may be wound on a winding tool prior to placement within a lumen of a needle. The wound suture knots may then be loading into the lumen (e.g., inner diameter) of the needle and/or may be pushed off of the winding tool. The suture knots may be pushed down against a pusher situated within the lumen of the needle.

Certain inventive features disclosed herein relate to the delivery of suture knots associated with certain heart valve repair systems and devices, and/or systems, process, and devices for repairing any other type of target organ tissue. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, or otherwise physically related to the second feature, element, component, device, or member.

The following includes a general description of human cardiac anatomy that is relevant to certain inventive features and examples disclosed herein and is included to provide context for certain aspects of the present disclosure. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

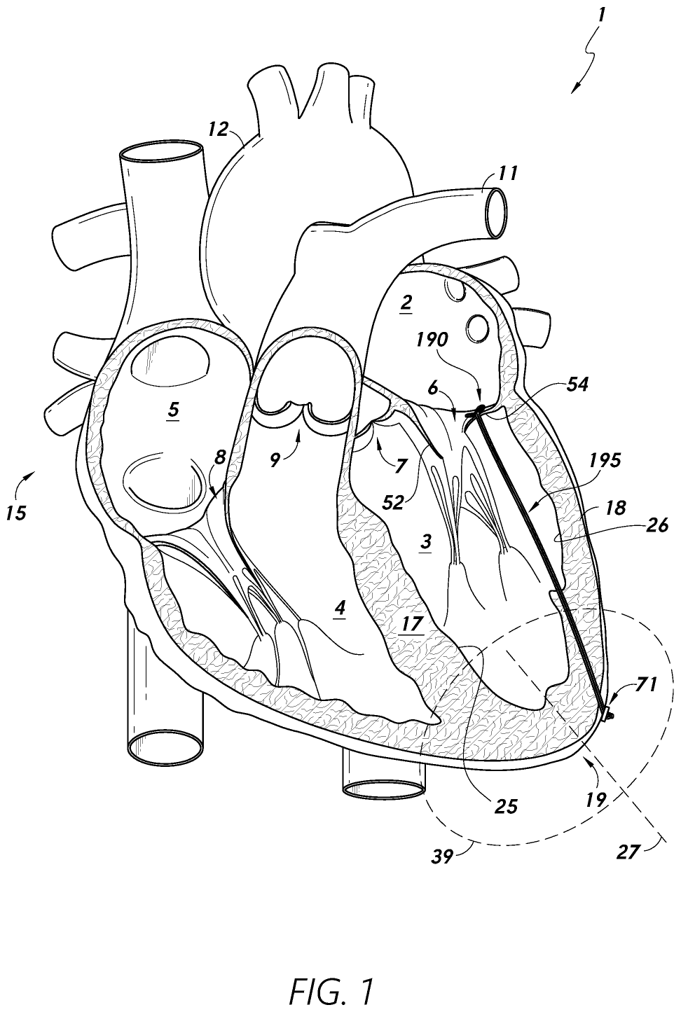

FIG. 1 illustrates an example representation of a heart 1 having various features relevant to certain aspects of the present inventive disclosure. The heart 1 includes four chambers, namely the left ventricle 3, the left atrium 2, the right ventricle 4, and the right atrium 5. A wall of muscle 17, referred to as the septum, separates the left 2 and right 5 atria and the left 3 and right 4 ventricles. The inferior tip 19 of the heart 1 is referred to as the apex and is generally located on the midclavicular line, in the fifth intercostal space. The apex 19 can be considered part of the greater apical region 39.

The left ventricle 3 is the primary pumping chamber of the heart 1. A healthy left ventricle is generally conical or apical in shape in that it is longer (along a longitudinal axis extending in a direction from the aortic valve 7 to the apex 19) than it is wide (along a transverse axis extending between opposing walls 25, 26 at the widest point of the left ventricle) and descends from a base 15 with a decreasing cross-sectional circumference to the point or apex 19. Generally, the apical region 39 of the heart is a bottom region of the heart that is within the left or right ventricular region but is distal to the mitral 6 and tricuspid 8 valves and toward the tip of the heart. More specifically, the apical region 39 may be considered to be within about 20 cm to the right or to the left of the median axis 27 of the heart 1.

The pumping of blood from the left ventricle is accomplished by a squeezing motion and a twisting or torsional motion. The squeezing motion occurs between the lateral wall 18 of the left ventricle and the septum 17. The twisting motion is a result of heart muscle fibers that extend in a circular or spiral direction around the heart. When these fibers contract, they produce a gradient of angular displacements of the myocardium from the apex 19 to the base 15 about the longitudinal axis of the heart. The resultant force vectors extend at angles from about 30-60 degrees to the flow of blood through the aortic valve 7. The contraction of the heart is manifested as a counterclockwise rotation of the apex 19 relative to the base 15, when viewed from the apex 19. A healthy heart can pump blood from the left ventricle in a very efficient manner due to the spiral contractility of the heart.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery ii and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The atrioventricular (e.g., mitral and tricuspid) heart valves may comprise a collection of chordae tendineae and papillary muscles for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve may comprise three leaflets and three corresponding papillary muscles (two shown in FIG. 1). The leaflets of the tricuspid valve may be referred to as the anterior, posterior and septal leaflets, respectively. The valve leaflets are connected to the papillary muscles by the chordae tendineae, which are disposed in the right ventricle 4 along with the papillary muscles.

Surrounding the ventricles (3, 4) are a number of arteries (not shown) that supply oxygenated blood to the heart muscle and a number of veins that return the blood from the heart muscle. The coronary sinus (not shown) is a relatively large vein that extends generally around the upper portion of the left ventricle 3 and provides a return conduit for blood returning to the right atrium 5. The coronary sinus terminates at the coronary ostium (not shown) through which the blood enters the right atrium.

With respect to the mitral valve 6, a normal mitral valve may comprise two leaflets (anterior and posterior) and two corresponding papillary muscles. The papillary muscles originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

Various disease processes can impair the proper functioning of one or more of the valves of the heart. These disease processes include degenerative processes (e.g., Barlow's disease, fibroelastic deficiency), inflammatory processes (e.g., rheumatic heart disease) and infectious processes (e.g., endocarditis). Additionally, damage to the ventricle from prior heart attacks (e.g., myocardial infarction secondary to coronary artery disease) or other heart diseases (e.g., cardiomyopathy) can distort the valve's geometry causing it to dysfunction. However, the vast majority of patients undergoing valve surgery, such as mitral valve surgery, suffer from a degenerative disease that causes a malfunction in one or more leaflets of the valve which results in prolapse and regurgitation.

The mitral valve 6 and tricuspid valve 8 can be divided into three parts: an annulus, leaflets, and a sub-valvular apparatus. The sub-valvular apparatus can be considered to include the papillary muscles and the chordae tendineae, which can elongate and/or rupture. If a valve is functioning properly, when closed, the free margins or edges of the leaflets come together and form a tight junction, the arc of which, in the mitral valve, is known as the line, plane or area of coaptation. Normal mitral and tricuspid valves open when the ventricles relax allowing blood from the atrium to fill the decompressed ventricle. When the ventricle contracts, the chordae tendineae advantageously properly tether or position the valve leaflets such that the increase in pressure within the ventricle causes the valve to close, thereby preventing blood from leaking into the atrium and assuring that substantially all of the blood leaving the ventricle is ejected through the aortic valve 7 or pulmonic valve 9 and into the arteries of the body. Accordingly, proper function of the valves depends on a complex interplay between the annulus, leaflets, and sub-valvular apparatus. Lesions in any of these components can cause the valve to dysfunction and thereby lead to valve regurgitation.

Generally, there are three mechanisms by which a heart valve becomes regurgitant or incompetent; they include Carpentier's type I, type II and type III malfunctions. A Carpentier type I malfunction involves the dilation of the annulus such that normally functioning leaflets are distracted from each other and fail to form a tight seal (e.g., do not coapt properly). Included in a type I mechanism malfunction are perforations of the valve leaflets, as in endocarditis. A Carpentier's type II malfunction involves prolapse of one or both leaflets above the plane of coaptation. This is the most common cause of mitral regurgitation and is often caused by the stretching or rupturing of chordae tendineae normally connected to the leaflet. A Carpentier's type III malfunction involves restriction of the motion of one or more leaflets such that the leaflets are abnormally constrained below the level of the plane of the annulus. Leaflet restriction can be caused by rheumatic disease (IIIa) or dilation of the ventricle (IIIb).

One or more chambers in the heart 1 may be accessed in accordance with certain heart valve-repair procedures and/or other interventions. Access into a chamber in the heart may be made at any suitable site of entry. In some implementations, access is made to a chamber of the heart, such as a target ventricle (e.g., left ventricle) associated with a diseased heart valve, through the apical region 39. For example, access into the left ventricle 3 (e.g., to perform a mitral valve repair) may be gained by making a relatively small incision at the apical region 39, close to (or slightly skewed toward the left of) the median axis 27 of the heart. Access into the right ventricle 4 (e.g., to perform a tricuspid valve repair) may be gained by making a small incision into the apical region 39, close to or slightly skewed toward the right of the median axis 27 of the heart. Accordingly, the ventricle can be accessed directly via the apex, or via an off-apex location that is in the apical region 39 but slightly removed from the tip/apex, such as via lateral ventricular wall, a region between the apex and the base of a papillary muscle, or even directly at the base of a papillary muscle. In some implementations, the incision made to access the appropriate ventricle of the heart is no longer than about 1 mm to about 5 cm, from 2.5 mm to about 2.5 cm, or from about 5 mm to about 1 cm in length. When a percutaneous approach is sought, no incision into the apex region of the heart may be made, but rather access into the apical region 39 may be gained by direct needle puncture, for instance by an 18-gauge needle, through which an appropriate repair instrument can be advanced.

### Heart Valve Leaflet Tethering

Certain inventive features disclosed herein relate to certain heart valve repair systems and devices, and/or systems, process, and devices for repairing any other type of target organ tissue. In some implementations, a tissue anchor delivery device may be employed in repairing a mitral valve in patients suffering from degenerative mitral regurgitation or other condition. In some implementations, a transapical, off-pump repair procedure is implemented in which at least part (e.g., a shaft portion/assembly) of a valve repair system is inserted in the left ventricle and advanced to the surface of the diseased portion of a target mitral valve leaflet and used to deploy/implant a tissue anchor in the target leaflet. The tissue anchor may advantageously be integrated or coupled with one or more artificial/synthetic cords serving a function similar to that of chordae tendineae. Such artificial cord(s) may comprise suture(s) and/or suture tail portions associated with a knot-type tissue anchor and may comprise any suitable or desirable material, such as expanded polytetrafluoroethylene (ePTFE) or the like. The term “suture” is used herein according to its broad and ordinary meaning and may refer to any elongate cord, strip, strand, line, tie, string, ribbon, strap, or portion thereof, or other type of material used in medical procedures. One having ordinary skill in the art will understand that a wire or other similar material may be used in place of a suture. Furthermore, in some contexts herein, the terms “cord,” “chord,” “chordae,” and “suture” may be used substantially interchangeably. In addition, use of the singular form of any of the suture-related terms listed above, including the terms “suture” and “cord,” may be used to refer to a single suture/cord, or to a portion thereof, or to a plurality of suture/cords, such as a pair of suture/cord tails emanating from a single anchor, knot, form, device, or other structure or assembly. Where a suture knot or anchor is deployed on a distal side of a tissue portion, and where two suture portions extend from the knot/anchor on a proximal side of the tissue, either or both of the suture portions may be referred to as a “suture” or a “cord,” regardless of whether both portions are part of a unitary suture or cord or are separate.

Processes for repairing a target organ tissue, such as repair of mitral valve leaflets to address mitral valve regurgitation, can include inserting a tissue anchor delivery device, such as a delivery device as described in PCT Application No. PCT/US2012/043761, (published as WO 2013/003228, and referred to herein as “the '761 PCT Application”) and/or in PCT Application No. PCT/US2016/055170 (published as WO 2017/059426 and referred to herein as “the '170 PCT Application”), the entire disclosures of which are incorporated herein by reference, into a body and extending a distal end of the delivery device to a proximal side of the target tissue (e.g., leaflet).

The '761 PCT Application and the '170 PCT Application describe in detail methods and devices for performing non-invasive procedures to repair a cardiac valve, such as a mitral valve. Such procedures include procedures to repair regurgitation that occurs when the leaflets of the mitral valve do not coapt properly at peak contraction pressures, resulting in an undesired backflow of blood from the ventricle into the atrium. As described in the '761 PCT Application and the '170 PCT Application, after the malfunctioning cardiac valve has been assessed and the source of the malfunction verified, a corrective procedure can be performed. Various procedures can be performed in accordance with the methods described therein to effectuate a cardiac valve repair, which may depend on the specific abnormality and the tissues involved.

With further reference to FIG. 1, FIG. 1 shows an example deployed leaflet/tissue anchor 190 deployed in a heart valve leaflet 54 and tethered to a heart/ventricle wall 18 via one or more sutures/suture tails 195 coupled to and/or associated with the anchor 190. The suture tails 195 coupled to the anchor 190 may be secured at the desired tension using a pledget 71 or other suture-fixing/locking device or mechanism on the outside of the heart wall 18 through which the suture tails 195 may run. A knot or other suture fixation mechanism or device may be implemented to hold the sutures at the desired tension and to the pledget 71. With the suture tail(s) 195 fixed to the ventricle wall 18, a portion of the suture tail(s) 195 disposed within the ventricle 3 may advantageously function as replacement leaflet cords (e.g., chordae tendineae) that are configured to tether the target leaflet 54 in a desired manner.

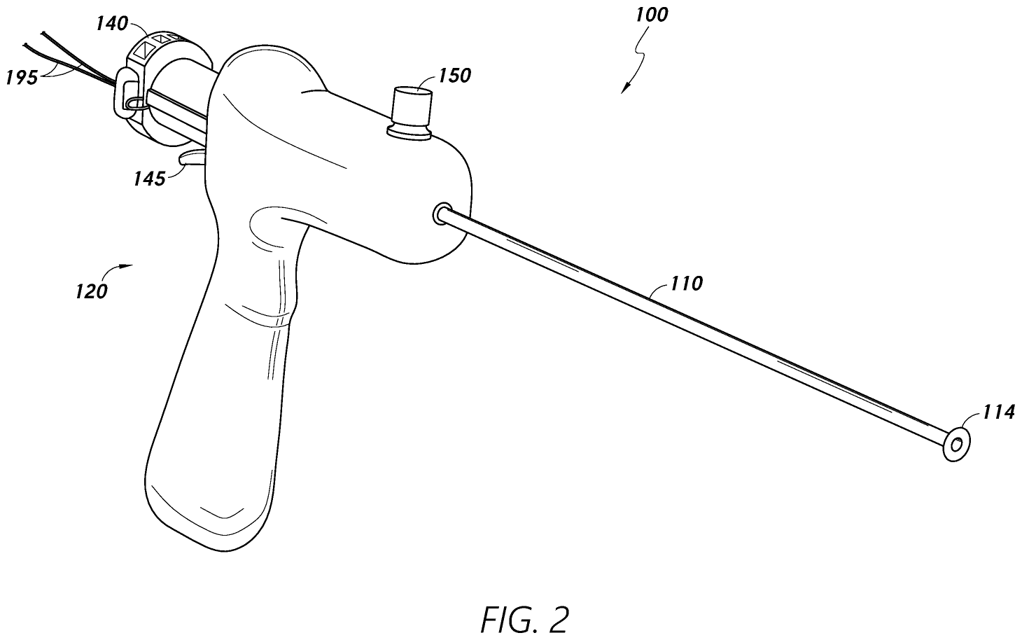

FIG. 2 is a perspective view of a tissue anchor delivery device in accordance with one or more examples. The tissue anchor delivery system 100 may be used to repair a heart valve, such as a mitral valve, and improve functionality thereof. For example, the tissue anchor delivery system 100 may be used to reduce the degree of mitral regurgitation in patients suffering from mitral regurgitation caused by, for example, midsegment prolapse of valve leaflets as a result of degenerative mitral valve disease. In order to repair such a valve, the tissue anchor delivery system 100 may be utilized to deliver and anchor tissue anchors, such as malleable tissue anchors, in a prolapsed valve leaflet. As described in detail below, such procedure may be implemented on a beating heart.

The delivery system 100 includes a rigid elongate tube no forming at least one internal working lumen. Although described in certain examples and/or contexts as comprising a rigid elongate tube, it should be understood that tubes, shafts, lumens, conduits, and the like disclosed herein may be either rigid, at least partially rigid, at least flexible, and/or at least partially flexible. Therefore, any such component described herein, whether or not referred to as rigid herein should be interpreted as possibly being at least partially flexible. In accordance with the present disclosure, the rigid elongate tube no may be referred to as a shaft for simplicity. Implementation of a valve-repair procedure utilizing the delivery system 100 can be performed in conjunction with certain imaging technology designed to provide visibility of the shaft no of the delivery system 100 according to a certain imaging modality, such as echo imaging. Generally, when performing a valve-repair procedure utilizing the tissue anchor delivery system 100, the operating physician may advantageously work in concert with an imaging technician, who may coordinate with the physician to facilitate successful execution of the valve-repair procedure.

In addition to the delivery shaft no, the delivery system wo may include a plunger feature 140. The tissue anchor delivery system 100 may further include a plunger lock mechanism 145, which may serve as a safety lock that locks the valve delivery system until ready for use or deployment of a leaflet anchor as described herein. The plunger 140 may have associated therewith a suture-release mechanism, which may be configured to lock in relative position a pair of suture tails 195 associated with a pre-formed knot anchor (not shown) to be deployed. For example, the suture portions 195 may be ePTFE sutures. The system wo may further comprise a flush port 150, which may be used to de-air the lumen of the shaft no. For example, heparinized saline flush, or the like, may be connected to the flush port 150 using a female Luer fitting to de-air the valve repair system 100. The term “lumen” is used herein according to its broad and ordinary meaning, and may refer to a physical structure forming a cavity, void, pathway, or other channel, such as an at least partially rigid elongate tubular structure, or may refer to a cavity, void, pathway, or other channel, itself, that occupies a space within an elongate structure (e.g., a tubular structure). Therefore, with respect to an elongate tubular structure, such as a shaft, tube, or the like, the term “lumen” may refer to the elongate tubular structure and/or to the channel or space within the elongate tubular structure.

The lumen of the shaft no may house a needle (not shown) configured to deploy one or more anchors, as described in detail herein. In some examples, the shaft no presents a relatively low profile. For example, the shaft no may have a diameter of approximately 3 mm or less (e.g., 9 Fr). The shaft no is associated with an atraumatic tip 114 feature. The atraumatic tip 114 can be an echogenic leaflet-positioner component, which may be used for deployment and/or positioning of the suture-type tissue anchor. The atraumatic tip 114, disposed at the distal end of the shaft no, may be configured to have deployed therefrom one or more valve anchors, as described herein.

The atraumatic tip 114 may be referred to as an “end effector.” In addition to one or more valve anchors and associated needle, the shaft no may house an elongated knot pusher tube (not shown; also referred to herein as a “pusher”), which may be actuated using the plunger 140 in some examples. As described in further detail below, the tip 114 provides a surface against which the target valve leaflet may be held in connection with deployment of a leaflet anchor.

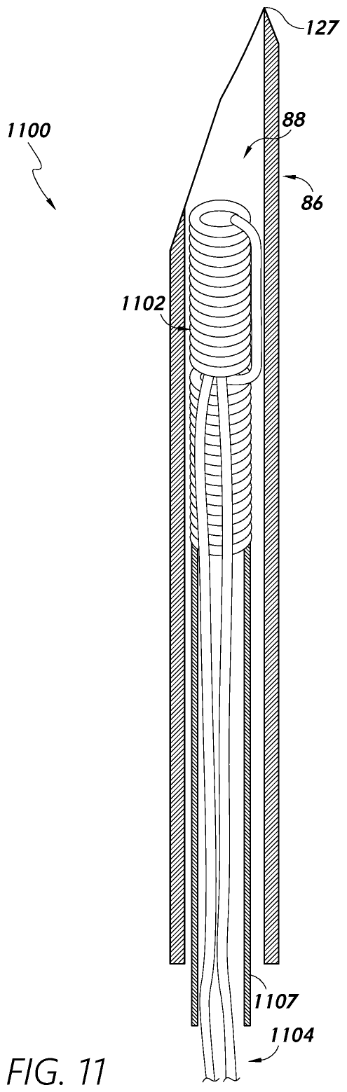

The delivery system 100 may be used to deliver any of the various tissue anchors described in greater detail below. For example, the delivery system 100 may be utilized to deliver a tissue anchor on a distal side of a mitral valve leaflet. The tip 114 (e.g., end effector), can be placed in contact with the ventricular side of a leaflet of a mitral valve. The tip 114 can be coupled to the distal end portion of the shaft no, wherein the proximal end portion of the shaft no may be coupled to a handle portion 120 of the delivery system 100, as shown. Generally, the elongate pusher (not shown) may be movably disposed within a lumen of the shaft no and coupled to a pusher hub (not shown) that is movably disposed within the handle 120 and releasably coupled to the plunger 140. A needle and/or catheter carrying one or more tissue anchors can be movably disposed within a lumen of the pusher and coupled to a needle hub (not shown) that is also coupled to the plunger 140. In some instances, the needle and/or catheter may comprise a pointed tip to facilitate puncturing a valve leaflet. However, in some instances, one or more tissue anchors may have pointed tips and/or the needle and/or catheter may not comprise a pointed tip. The plunger 140 can be used to actuate or move the needle and/or the pusher during deployment of a distal anchor (see, e.g., FIG. 10) and is movably disposed at least partially within the handle 120. For example, the handle 120 may define a lumen in which the plunger 140 can be moved. During operation, the pusher may also move within the lumen of the handle 120. The plunger lock 145 can be used to prevent the plunger 140 from moving within the handle 120 during storage and prior to performing a procedure to deploy a tissue anchor.

The needle and/or catheter may have the one or more tissue anchors disposed at a distal portion thereof while maintained in the shaft no. For example, one or more tissue anchors may be arranged generally longitudinally while within a lumen of the needle and/or catheter. In some instances, one or more suture tails may extend from each of the one or more tissue anchors. The suture tails 195 may extend through the lumen of the needle and/or through a passageway of the plunger 140 and may exit the plunger 140 at a proximal end portion thereof.

The delivery device/system 100 can further include a suture/tether catch mechanism (not shown) coupled to the plunger 140 at a proximal end of the delivery system 100, which may be configured to releasably hold or secure a suture 195 extending through the delivery system 100 during delivery of a tissue anchor as described herein. The suture catch can be used to hold the suture 195 with a friction fit or with a clamping force and can have a lock that can be released.

As described herein, the anchor delivery system 100 can be used in beating heart mitral valve repair procedures. In some instances, the shaft 110 of the delivery system 100 can be configured to extend and contract with the beating of the heart. During systolic contraction, the median axis of the heart generally shortens. For example, with reference to FIG. 1, the distance from the apex 19 of the heart to the valve leaflets 52, 54 can vary by about 1 cm to about 2 cm with each heartbeat in some patients. In some instances, the length of the shaft 110 that protrudes from the handle 120 can change with the length of the median axis of the heart. That is, distal end of the shaft 110 can be configured to be floating such that the shaft can extend and retract with the beat of the heart so as to maintain contact with the target mitral valve leaflet.

Advancement of the delivery system 100 may be performed in conjunction with echo imaging, direct visualization (e.g., direct transblood visualization), and/or any other suitable remote visualization technique/modality. With respect to cardiac procedures, for example, the delivery system 100 may be advanced in conjunction with transesophageal (TEE) guidance and/or intracardiac echocardiography (ICE) guidance to facilitate and to direct the movement and proper positioning of the device for contacting the appropriate target cardiac region and/or target cardiac tissue (e.g., a valve leaflet, a valve annulus, or any other suitable cardiac tissue). Typical procedures that can be implemented using echo guidance are set forth in Suematsu, Y., J. Thorac. Cardiovasc. Surg. 2005; 130:1348-56 (“Suematsu”), the entire disclosure of which is incorporated herein by reference.

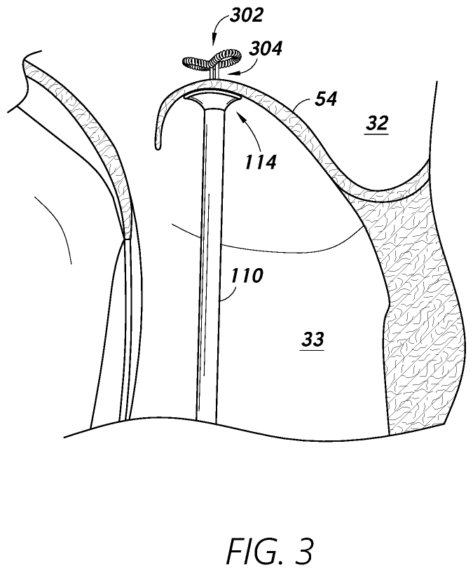

FIG. 3 shows the shaft 110 of the tissue anchor delivery system positioned on the target valve leaflet 54 (e.g., mitral valve leaflet). For example, the target site of the valve may be slowly approached from the ventricle side thereof by advancing the distal end of the shaft 110 along or near to the posterior wall of the ventricle 33 (e.g., left ventricle) without contacting the ventricle wall. Successful targeting and contacting of the target location on the leaflet 54 can depend at least in part on accurate visualization of the shaft 110 and/or tip/end effector 114 throughout the process of advancing the tip 114 to the target site. Generally, echocardiographic equipment may be used to provide the necessary or desired intra-operative visualization of the shaft 110 and/or tip 114.

Once the tip 114 is positioned in the desired position, the distal end of the shaft 110 and the tip 114 may be used to drape, or “tent,” the leaflet 54 to better secure the tip 114 in the desired position, as shown in FIG. 3. Draping/tenting may advantageously facilitate contact of the tip 114 with the leaflet 54 throughout one or more cardiac cycles, to thereby provide more secure or proper deployment of leaflet anchor(s). The target location may advantageously be located relatively close to the free edge of the target leaflet 54 to minimize the likelihood of undesirable intra-atrial wall deployment of the anchor. Navigation of the tip 114 to the desired location on the underside of the target valve leaflet 54 may be assisted using echo imaging, as described in detail herein. Echo imaging may be relied upon to confirm correct positioning of the tip 114 prior to anchor/knot deployment.

With the shaft 110 positioned against the target leaflet 54, the plunger of the tissue anchor delivery system 100 can be actuated to move the one or more needles and/or one or more pushers disposed within the one or more needles, such that the coiled sutureform portions of the suture anchors extend upward within and/or out of the lumens of the one or more anchors. As the plunger is actuated, distal piercing portions of the needles puncture the leaflet 54 and form one or more openings in the leaflet. In some instances, a needle may be configured to be projected a distance of between about 5-8 mm (about 0.2-0.3 inches), or less, distally beyond the distal end of the shaft no (e.g., beyond the tip 114). In some instances, a needle may be projected a distance of between about 4-10 mm (about 0.15-0.4 inches). In some instances, a needle may be configured to extend until a distal tip of the needle (e.g., a pointed tip) extends at least partially through the leaflet 54. The needle and/or the distal tip of the needle may be configured to pierce and/or penetrate a leaflet of a heart valve. While one or more needles and/or sutureforms may be projected partially into the atrial side 32 of the leaflet 54, the shaft 110 and/or tip 114 may be advantageously configured to remain entirely on the ventricular side 33 of the leaflet 54.

As one or more pushers (not shown) within the tissue anchor delivery device shaft 110 are moved distally, distal ends of the pushers may advantageously move and/or push the distal coiled sutureforms (e.g., pre-deployment coiled portions of the suture anchors 302) out of and/or beyond the distal ends of the needles and further within the atrium 32 of the heart on a distal side of the leaflet 54, such that the sutureforms extend distally beyond distal ends of the needles. The pushers may be configured to press against the sutureforms. In some instances, an entire coiled sutureform may be configured to extend beyond a distal end of a needle.

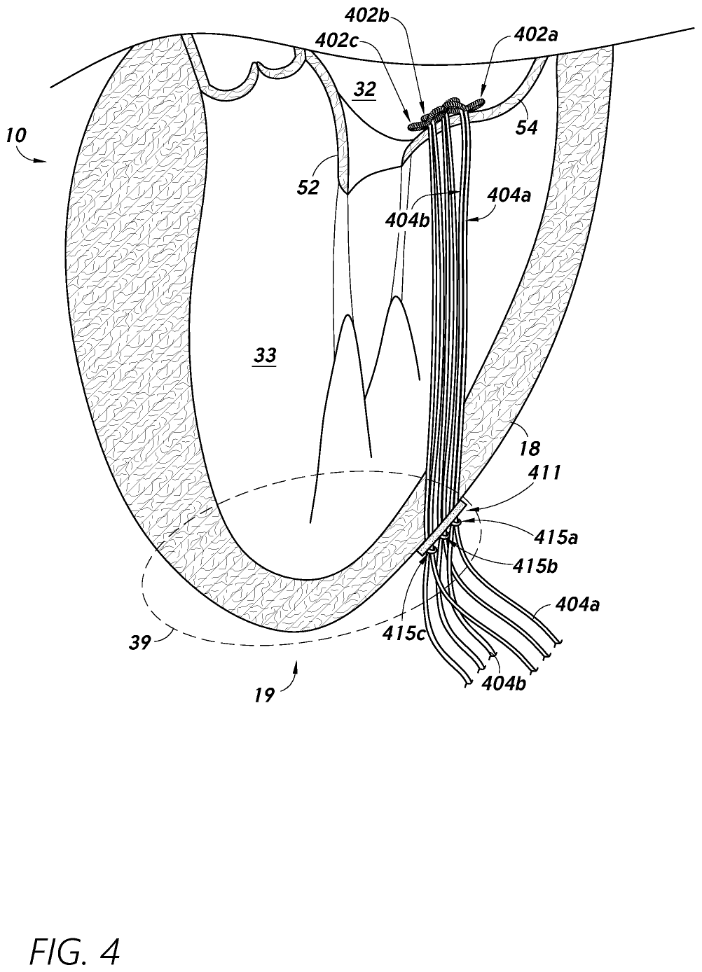

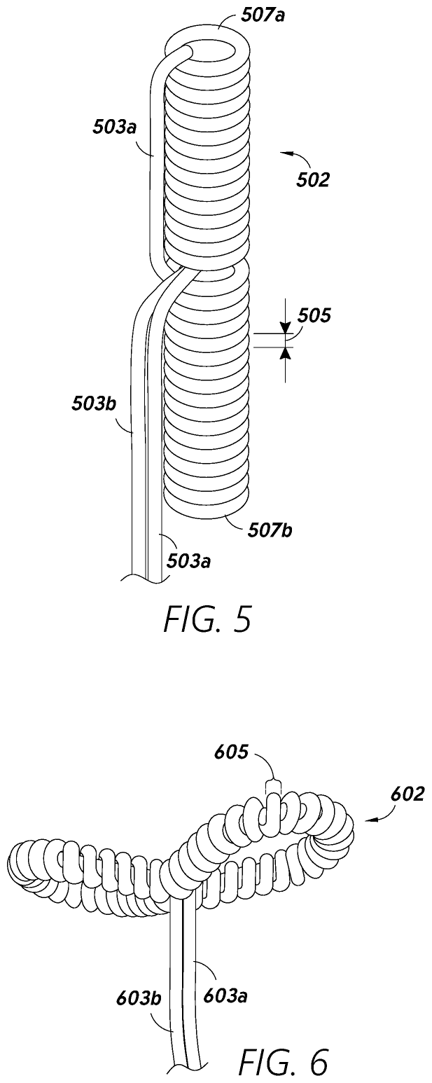

As shown in FIG. 3, after a sutureform has been pushed off and/or removed from a needle, pulling one or more of the suture tails 304 (e.g., suture strands extending from the coiled portion of the suture) associated with the tissue anchor 302 proximally can cause the sutureform to form a bulky knot anchor 302. For example, the bulky knot suture anchor 302 may be formed by approximating opposite ends of the coils of the sutureform (see FIG. 5) towards each other to form one or more loops. After the sutureform has been formed into the bulky knot 302, the shaft 110 can be withdrawn proximally, leaving the tissue anchor 302 disposed on the distal atrial side of the leaflet 54, as shown in FIG. 4. In some instances, two suture tails 304 for each bulky knot 302 may extend from the proximal/ventricle side 33 of the leaflet 54 and out of the heart 1. For example, the delivery device shaft 110 can be slid/withdrawn over the suture tail(s) 304.

It may be desirable to advance the shaft 110 within the ventricle 33 in such a way as to avoid traversing areas populated by papillary muscles and/or associated chordae tendineae to avoid entanglement therewith. In order to facilitate or ensure avoidance of such anatomy, imaging technology may advantageously be implemented to provide at least partial visibility of the shaft 110 within the ventricle 33, as well as certain anatomical features within the ventricle. With respect to visibility of the shaft 110 in the ventricle 33, echogenic characteristics of the shaft 110 can affect the visibility thereof using echo imaging modalities. Therefore, a shaft 110 having relatively high echogenicity as described in detail herein may advantageously allow for more accurate and/or simplified advancement of the shaft 110, as well as placement of the tip 114 at the target implantation site at the valve leaflet 54 (e.g., an anterior or posterior leaflet of a mitral valve). In some implementations, hybrid imaging technologies may be used, wherein echo imaging is used in combination with a different imaging modality. Multi-imaging modalities may provide improved visibility of anatomical and/or delivery system components.

FIG. 4 shows a cutaway view of multiple deployed leaflet anchors 402a, 402b, 402c in accordance with one or more instances of the present disclosure. While FIG. 4 shows three deployed leaflet anchors 402, any number of leaflet anchors may be deployed. A first leaflet anchor 402a, second leaflet anchor 402b, and/or third leaflet anchor 402c may be delivered via the delivery shaft 110 simultaneously or sequentially. In some instances, the leaflet anchors 402 may each be delivered via the same delivery shaft 110 without requiring removal of the delivery shaft 110. In other words, the delivery shaft 110 may be configured to be delivered into the ventricle 33 and/or into contact with the leaflet 54 and deploy multiple leaflet anchors 402 before removal of the delivery shaft 110 from the ventricle 33 and/or leaflet 54. In some instances, each of the leaflet anchors 402 may be configured to be delivered via a separate lumen of the delivery shaft 110 and/or a separate needle. Moreover, each of the leaflet anchors 402 may be delivered through separate holes created in the leaflet 54.

The suture tails 404 coupled to the anchors 402 may be secured at the desired tension using a pledget 411 or other suture-fixing/locking device or mechanism on the outside of the heart through which the suture tails 404 may run. For example, a first suture tail 404a and/or a second suture tail 404b of a first leaflet anchor 402a may form a first knot 415a, one or more suture tails 404 of a second leaflet anchor 402b may form a second knot 415b, and/or one or more suture tails 404 of a third leaflet anchor 402c may form a third knot 415c to secure the leaflet anchors 402 to the pledget 411. While multiple leaflet anchors 402 may be secured using a single pledget 411 as shown in FIG. 4, multiple pledgets 411 may alternatively be used. For example, each knot 415 may secure to a separate pledget 411. The knot 415 and/or other suture fixation mechanism and/or device may be implemented to hold the sutures 404 at the desired tension and/or to the pledget 411. With the suture tail(s) 404 fixed to the ventricle wall 18, a ventricular portion of the suture tail(s) 404 (e.g., the portion of the suture tail 404 that is within the ventricle 33) may advantageously function as replacement leaflet cords (e.g., chordae tendineae) that may be configured to tether the target leaflet 54 in a desired manner. As shown in FIG. 4, the pledget 411 and/or one or more knots 415 may be configured to be situated at least partially external to the ventricle 33.

In certain instances, the pledget 411 may be a low-porosity and/or relatively stiff pledget. Such a pledget 411 may advantageously allow for the desired tension of the suture tails 404 to be sustained over an extended post-operative period of time. In some instances, suture tying and/or fixation may be implemented using one or more soft tissue retractors and/or right-angle clamps, which may have rubber shods associated therewith to reduce the risk of damage to the replacement cords.

In certain implementations, testing of location and/or tension of the anchors 402 and/or suture tail(s) 404 may be performed by gently tensioning the suture tails 404 until leaflet motion is felt and/or observed. Echo imaging technology may be used to view and verify the anchor placement and resulting leaflet function. The steps and processes outlined above for placing one or more suture-knot-type tissue anchors may be repeated as necessary until the desired number of anchors 402 have been implanted on the target valve leaflet 54 and/or at another leaflet 52. In some implementations, tension adjustment in the suture tail(s)/cord(s) 404 associated with multiple leaflet anchors 402 may be performed simultaneously. The appropriate number of leaflet anchors 402 may advantageously be determined to produce the desired coaptation of the target valve leaflets 54, 52. All deployed leaflet anchors 402 may advantageously be below the surface of coaptation. With respect to posterior mitral valve leaflet repair, the anterior leaflet 52 may advantageously touch the posterior leaflet 54 basal to the leaflet anchor(s) 402. The pledget 411 may be drawn against the epicardial surface, and all the suture tails/cords 404 may be inserted through a tourniquet so that all cords can be tensioned to the desired effective coaptation together.

In some implementations, one or more leaflet anchors 402 may be deployed in each of the mitral valve leaflets 52, 54, and/or sutures/cords 404 coupled to separate leaflets 52, 54 may be secured together in the heart by tying them together with knots and/or by another suitable attachment device, creating an edge-to-edge repair to decrease the septal-lateral distance of the mitral valve orifice.

### Suture Anchors

FIG. 5 illustrates an example suture anchor 502 in accordance with one or more examples. In some examples, the suture anchor 502 may comprise one or more sutures formed into a sutureform. A suture anchor 502 formed at least partially of a suture may be referred to herein as a “sutureform” or “suture knot.” A sutureform may comprise an at least partially coiled suture comprising one or more coils 505. The sutureform may be configured to be formed into a knot (e.g., a “bulky knot”) as shown in FIG. 6. One or more tail portions 503 of the suture anchor 502 may be configured to extend from and/or may be an extension (e.g., a free end) of the sutureform and/or knot to attach to, interconnect with, and/or interlock with another sutureform, knot, and/or other type of suture anchor 502. Accordingly, one or more tail portions 503 may form tethering sutures between multiple suture anchors 502.

In some examples, a suture forming one or more suture anchors 502 may also serve as a tethering suture between multiple suture anchors 502. For example, a first (e.g., distal) suture anchor 502 may be at least partially formed of a suture having two tail portions 503 (e.g., a first tail portion 503a and a second tail portion 503b) which may be configured to attach to, extend into, and/or secure to a second (e.g., proximal) suture anchor. One or more tail portions 503 extending from the first suture anchor 502 may be configured to fit through one or more coils 505 of the suture anchor 502 and/or another suture anchor. For example, at least a portion of a second suture anchor 502 may be wrapped around one or the one or more tail portions 503 extending from the first suture anchor 502. The first suture anchor 502 may be coiled to form an internal lumen through the one or more coils/windings 505 of the suture anchor. Additional suture anchors may additionally comprise one or more tail portions 503 which may be configured to extend out of the sutureform and/or knot of the additional suture anchors. In some examples, the one or more tail portions 503 extending from each suture anchor 502 (e.g., the first tail portion 503a and/or second tail portion 503b) may at least partially form additional suture anchors. In other words, the first suture anchor 502 and/or additional suture anchors may be formed from a common set of one or more sutures (e.g., a single suture).

In the example shown in FIG. 5, the first tail portion 503a may extend from a distal end soya of the suture anchor 502 and/or may be configured to pass along the suture anchor 502 external to all of the one or more coils 505 of the suture anchor 502 and/or beyond a proximal end 507b of the suture anchor 502. The second tail portion 503b may extend directly from the proximal end 507b of the suture anchor 502.

In some instances, the first tail portion 503a may be configured to pass between coils 505 of the suture anchor 502. The first tail portion 503a may be configured to interlock with a second tail portion 503b at a midpoint of the suture anchor 502. In some instances, the second tail portion 503b may be configured to exit from an internal lumen of the suture anchor 502 at a midpoint of the suture anchor 502 (e.g., between two coils 505 of the suture anchor 502) and/or may interlock with the first tail portion 503a. For example, the first tail portion 503a may be twisted with the second tail portion 503b to form a secure attachment between the first tail portion 503a and the second tail portion 503b at a midpoint of the suture anchor 502. In some instances, the first tail portion 503a and/or the second tail portion 503b may be configured to form one or more coils 505 of the suture anchor 502. The second tail portion 503b may be configured to pass along an exterior of at least a portion of the suture anchor 502 (e.g., along one or more coils 505 at or near a proximal end 507b of the suture anchor 502) at or near the proximal end 507b of the suture anchor 502. In some instances, the first tail portion 503a and/or the second tail portion 503b may be configured to be twisted with each other and/or with one or more coils 505 of the suture anchor 502 to form a secure attachment to each other and/or to the coils 505 of the suture anchor 502.

FIG. 6 illustrates a suture knot (e.g., bulky knot) formed from a suture anchor (e.g., the suture anchor 502 of FIG. 5) in accordance with one or more instances. In some instances, a sutureform (e.g., suture anchor) may be configured to form a suture knot 602 when the sutureform is removed from a from a delivery device (e.g., a needle). The suture knot 602 formed by the sutureform may create one or more openings and/or loop portions. For example, the suture knot 602 may form a figure-8 pattern comprising two interlocking loop portions, as shown in FIG. 6. One or more tail portions 603 of the suture knot 602 may be configured to extend through one or more delivery devices (e.g., needles, shaft, tubes, and/or catheters) and/or may be accessible to surgeons. In some instances, one or more tail portions 603 from the suture knot 602 (e.g., the first tail portion 603a and/or second tail portion 603b) may be configured to form a tether between the suture knot 602 and a locking point (e.g., at a pledget and/or other device at a ventricle wall). Accordingly, when the one or more tail portions 603 of the suture knot 602 are cinched, the one or more tail portions 603 may be configured to reduce the distance between the suture knot 602 and a tethering point at a ventricle wall or otherwise. The suture knot 602 may comprise multiple windings 605 and/or coils. In some examples, the windings 605 may have a helical form and/or may form generally tubular cords of the suture knot 602.

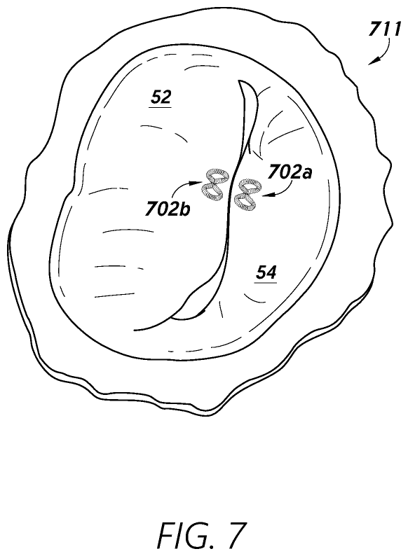

FIG. 7 illustrates an overhead view of an upper/distal side of a valve 711 (e.g., viewing the mitral valve from the left atrium) having two suture knots 702 with a bulky knot form delivered to the upper/distal side of the valve 711. As shown in FIG. 7, a first anchoring element 702a may be delivered to a first leaflet 54 and/or a second anchoring element 702b may be delivered to a second leaflet 52. However, any number of anchoring elements 702 may be delivered to either leaflet. For example, the first anchoring element 702a and second anchoring element 702b may alternatively both be delivered to the first leaflet 54.

### Suture Anchor Delivery Systems

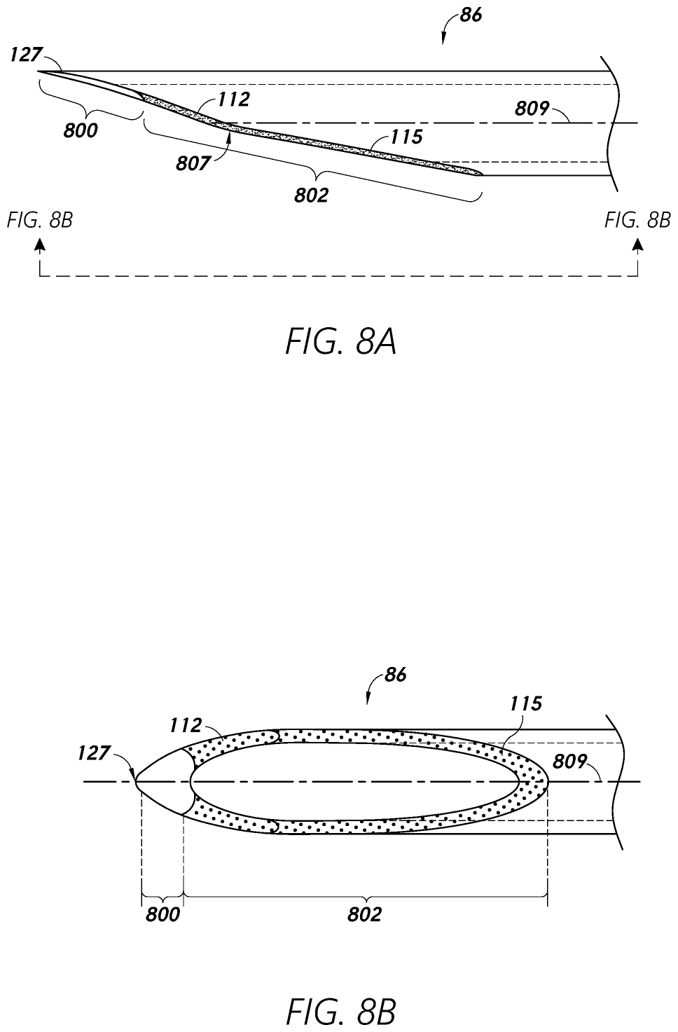

FIGS. 8A and 8B illustrate portions of example needles 86 configured for delivery of one or more suture anchors in accordance with one or more instances. The needle 86 may comprise an inner lumen configured to receive one or more suture anchors.

The needle 86 may comprise a tip 127 (e.g., a pointed end) configured to puncture and/or dilate one or more valve leaflets and/or other tissue. The configuration of the needle tip 127 may change the way the needle 86 penetrates through tissue and/or the cut area through the tissue once the needle 86 is removed. The needle 86 may comprise the tip 127, a distal beveled edge 112, and/or a proximal beveled edge 115. The tip 127 can be very sharp or pointed to facilitate initial penetration of the needle 86 through the tissue. A portion 800 of the distal beveled edge 112 can have sharp cutting surfaces. A remainder of the distal beveled edges 112 and proximal beveled edges 115 have smoothed, non-cutting surfaces. The portion 802 (e.g., the non-sharp surface of the beveled edge 112 and the beveled edge 112) can be smoothed in a wide variety of different ways. For example, the portion 802 can be polished in any conventional manner. In one exemplary instance, the portion 802 may be smooth, with non-cutting electropolished surfaces. The non-cutting surfaces may be configured to stretch the tissue rather than cut the tissue. Thus, non-cutting surfaces of the needle 86 can result in a reduced size puncture hole when the needle 86 is removed. The stretched tissue can return to its original size or close to its original size.

The distal beveled portion 112 may be formed from multiple (e.g., two) beveled cuts, each deflecting away from the tip 127. Furthermore, the portion 800 of the distal beveled portion 112 may advantageously be relatively sharp compared to the radiused edges/surfaces of the proximal beveled surface 115 and the radiused portion 802 of the distal beveled surface/portion 112. The distal 112 and proximal 115 beveled portion may be separated by an inflection point 807, which may be aligned with or near the central axis 809 of the needle 86. The radiused portion 802 of the needle may induce stretching of the tissue rather than cutting tissue. For example, the radiused portion 802 may cause the needle puncture to be dilated during the insertion of the needle and deployment of the pledget, suture knot, memory metal wire anchor, or other type of distal anchor. When the needle is removed, the dilated tissue may relax back to its previous form to result in a relatively smaller puncture dimension.

With only the portion 800 of the distal beveled portion 112 remaining un-radiused and relatively sharp, the tissue cut area may be relatively smaller than for needles having distal penetration portions that are not radiused. Furthermore, tissue stretch area may be maximized or relatively larger. The sharp portion 800, however, may promote ease of tissue penetration when cutting through tissue during initial needle penetration. In some instances, only a portion of the needle 86 (e.g., the portion 800) may extend beyond a distal side of a valve leaflet to allow delivery of one or more suture anchors from a lumen of the needle 86.

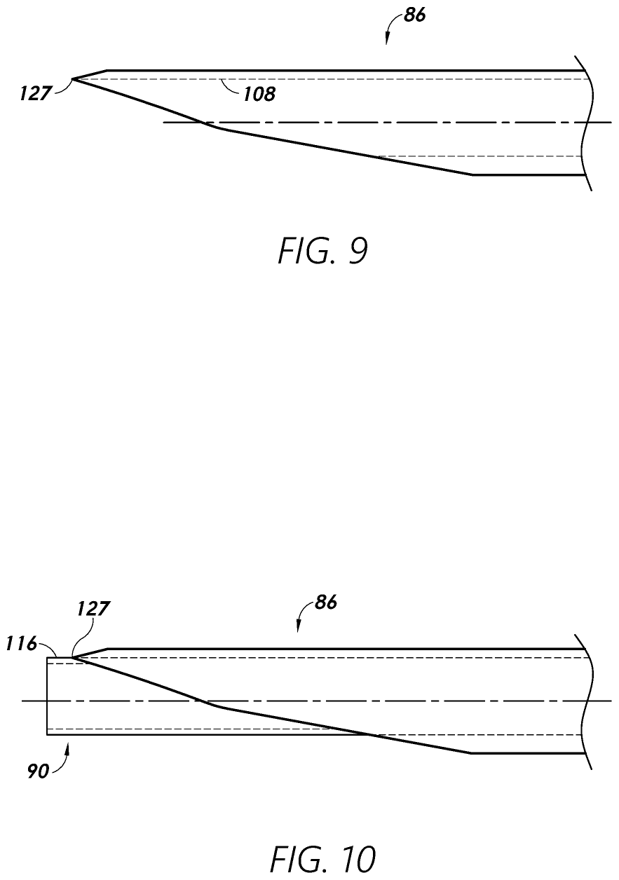

FIG. 9 illustrates another exemplary instance of a needle 86. In the example illustrated by FIG. 9, the sharp tip 127 of the needle 86 is coincident with an interior lumen wall 108 of the needle 86. FIG. 10 illustrates alignment of a pusher 90 extending through the lumen of the needle 86 with respect to the sharp tip 127 of the needle 86. A position of the sharp tip 127 may be adjacent to an outside surface 116 of the pusher 90. The pusher 90 may be composed of any suitable material(s), which can include stainless steel and/or polyetheretherketone (PEEK).

### Intra-Lumen Suture Knot Deployment

FIG. 11 illustrates an example delivery system 1100 for delivering and/or deploying a suture anchor 1102 from within a lumen 88 of a needle 86 in accordance with one or more instances. The needle 86 may be at least partially composed of one or more metals (e.g., stainless steel) and/or other suitable materials and/or may have a minimal width to limit a profile of the needle 86. For example, the needle 86 may be a 16-gauge hypo tube. The needle 86 may comprise one or more sharp and/or cutting edges and/or one or more edges of the needle 86 may be finished and/or rounded such that the cutting edges of the needle 86 may be configured to promote tissue dilation rather than cutting of tissue. For example, the edges of the needle 86 at or near the tip 127 of the needle 86 may be electropolished. In some instances, the tip 127 may extend along an inner diameter of the needle 86 as opposed to an outer diameter of the needle 86 to avoid snagging on the suture anchor 1102.

One or more pushers 1107 may be used to press against the suture anchor 1102 situated within the needle 86. In some instances, a pusher 1107 may comprise a hypo tube and/or similar device having a diameter that is less than the inner diameter of the needle 86 such that the pusher 1107 can fit and/or maneuver within the lumen 88 of the needle 86. The pusher 1107 may have a hollow interior and/or lumen configured to receive one or more suture tails 1104 extending from the suture anchor 1102 delivered via the needle 86. In some instances, suture tails 1104 extending through the pusher 1107 may be accessible to a surgeon. For example, the suture tails 1104 may be pulled to modify and/or tighten the suture anchors (e.g., to transform the suture anchor 1102 into a bulky knot and/or to press the bulky knot against a leaflet). In some instances, pulling on the suture tails 1104 may press the suture anchor 1102 against the pusher 1107, which may cause the suture anchor 1102 to form a bulky knot.

In some instances, the pusher 1107 may have a generally blunt leading edge (e.g., an edge configured to contact and/or press against the suture anchor 1102). The leading edge may comprise a blunt finish to prevent damage to the suture anchors and/or suture tails 1104. The pusher 1107 may be composed of any suitable material(s), which can include stainless steel.

While a single suture anchor 1102 is shown within the needle 86 in FIG. 11, the needle 86 may be configured to carry and/or deploy any number of suture anchors and/or similar devices. The suture anchor 1102 may be composed of any suitable material(s), which can include ePTFE, polyethylene terephthalate (PET) (e.g., ETHIBOND EXCEL braided polyester suture, Ethicon), monofilament, and/or multifilament. The suture anchor 1102 may be configured to be wound (e.g., on a knot winding tool) and/or woven into a tubular configuration prior to being loaded into the needle 86. In some instances, a suture anchor 1102 may comprise a combination of sutures and/or tubes. The suture anchor 1102 may be at least partially composed of one or more porous materials to enhance cell growth and/or assimilation into surrounding tissue.

While only a single needle 86 is shown in FIG. 11, multiple needles 86 carrying one or more suture anchors may be utilized simultaneously. For example, multiple needles 86 may be configured for delivery via a single shaft and/or catheter.

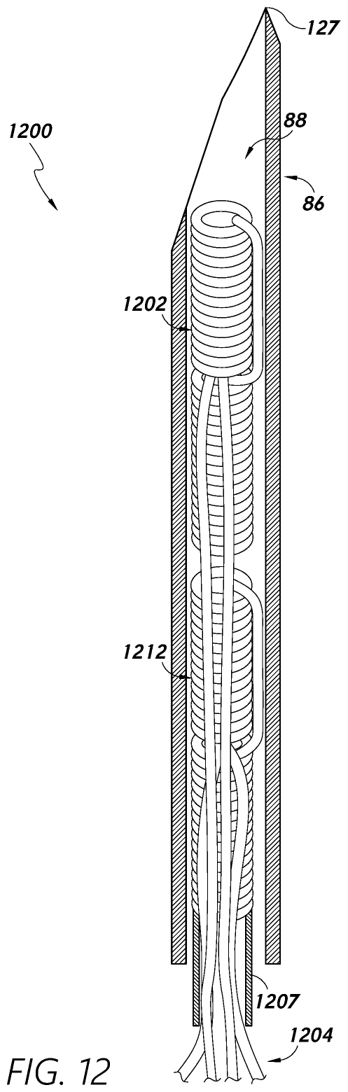

FIG. 12 illustrates an example delivery system 1200 for delivering and/or deploying one or more suture anchors from within a lumen 88 of a needle 86 in accordance with one or more instances. The needle 86 may be at least partially composed of one or more metals (e.g., stainless steel) and/or other suitable materials and/or may have a minimal width to limit a profile of the needle 86. For example, the needle 86 may be a 16-gauge hypo tube. The needle 86 may comprise one or more sharp and/or cutting edges and/or one or more edges of the needle 86 may be finished and/or rounded such that the cutting edges of the needle 86 may be configured to promote tissue dilation rather than cutting of tissue. For example, the edges of the needle 86 at or near the tip 127 of the needle 86 may be electropolished.

One or more pushers 1207 may be used to press against one or more suture anchors situated within the needle 86. In some instances, a pusher 1207 may comprise a hypo tube and/or similar device having a diameter that is less than the inner diameter of the needle 86 such that the pusher 1207 can fit and/or maneuver within the lumen 88 of the needle 86. The pusher 1207 may have a hollow interior and/or lumen configured to receive one or more suture tails 1204 extending from a first suture anchor 1202 and/or a second suture anchor 1212 delivered via the needle 86. In some instances, suture tails 1204 extending through the pusher 1207 may be accessible to a surgeon. For example, the suture tails 1204 may be pulled to modify and/or tighten the suture anchors (e.g., to transform the suture anchors into bulky knots and/or to press the bulky knots against a leaflet).

In some instances, the pusher 1207 may have a generally blunt leading edge (e.g., an edge configured to contact and/or press against the first suture anchor 1202 and/or the second suture anchor 1212). The leading edge may comprise a blunt finish to prevent damage to the suture anchors and/or suture tails 1204. The pusher 1207 may be composed of any suitable material(s), which can include stainless steel.

While two suture anchors are shown within the needle 86 in FIG. 12, the needle 86 may be configured to carry and/or deploy any number of suture anchors and/or similar devices. The first suture anchor 1202 and/or second suture anchor 1212 may be composed of any suitable material(s), which can include ePTFE, polyethylene terephthalate (PET) (e.g., ETHIBOND EXCEL braided polyester suture, Ethicon), monofilament, and/or multifilament. The first suture anchor 1202 and/or second suture anchor 1212 may be configured to be wound (e.g., on a knot winding tool) and/or woven into a tube configuration prior to being loaded into the needle 86. In some instances, a suture anchor may comprise a combination of sutures and/or tubes. The first suture anchor 1202 and/or second suture anchor 1212 may be at least partially composed of one or more porous materials to enhance cell growth and/or assimilation into surrounding tissue.

The first suture anchor 1202 and/or second suture anchor 1212 may be configured to be stacked end-to-end and/or lengthwise (e.g., longitudinally) within the needle 86. For example, as the pusher 1207 presses against the second suture anchor 1212 to press the second suture anchor 1212 upward and/or to a distal end of the needle 86, the second suture anchor 1212 may correspondingly be configured to translate force from the pusher 1207 to the first suture anchor 1202 by pressing the first suture anchor 1202 upwards and/or beyond the distal end of the needle 86. The first suture anchor 1202 and the second suture anchor 1212 may be stacked in series along a length of the needle 86.

The suture anchors may be configured to be sequentially deployed form the needle 86. For example, the first suture anchor 1202 may be situated nearer the distal end and/or opening of the needle 86 than the second suture anchor 1212. Accordingly, the first suture anchor 1202 may be pressed out of the needle 86 while the second suture anchor 1212 remains within the needle 86 and/or is repositioned near the distal end and/or opening of the needle 86. The second suture anchor 1212 may be configured to be delivered sequentially through the same opening through a leaflet as the first suture anchor 1202 and/or the needle 86 may be moved following deployment of the first suture anchor 1202 to deploy the second suture anchor 1212 at a different location and/or different opening through the leaflet and/or different leaflet.

While only a single needle 86 is shown in FIG. 12, multiple needles 86 carrying one or more suture anchors may be utilized simultaneously. For example, multiple needles 86 may be configured for delivery via a single shaft and/or catheter.

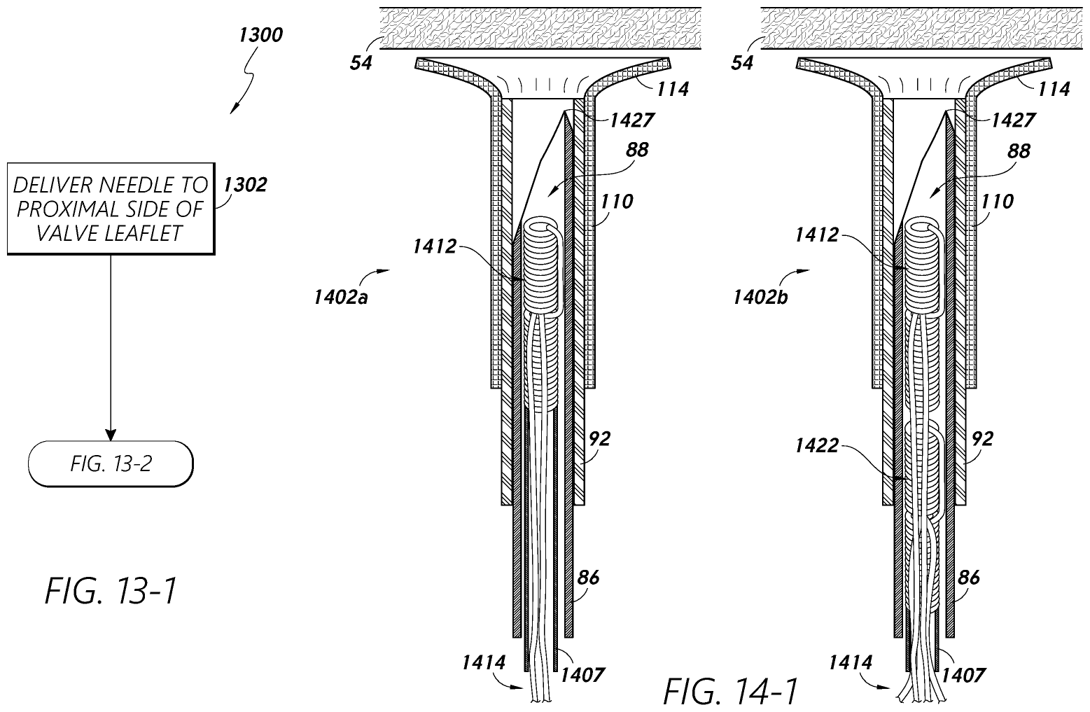

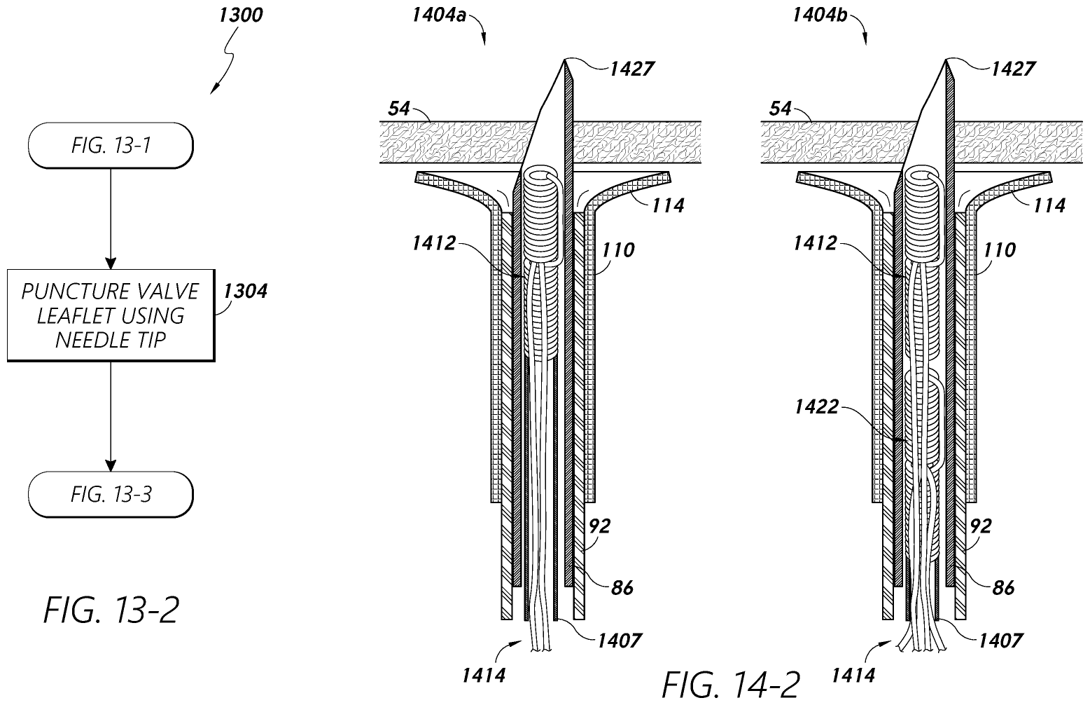

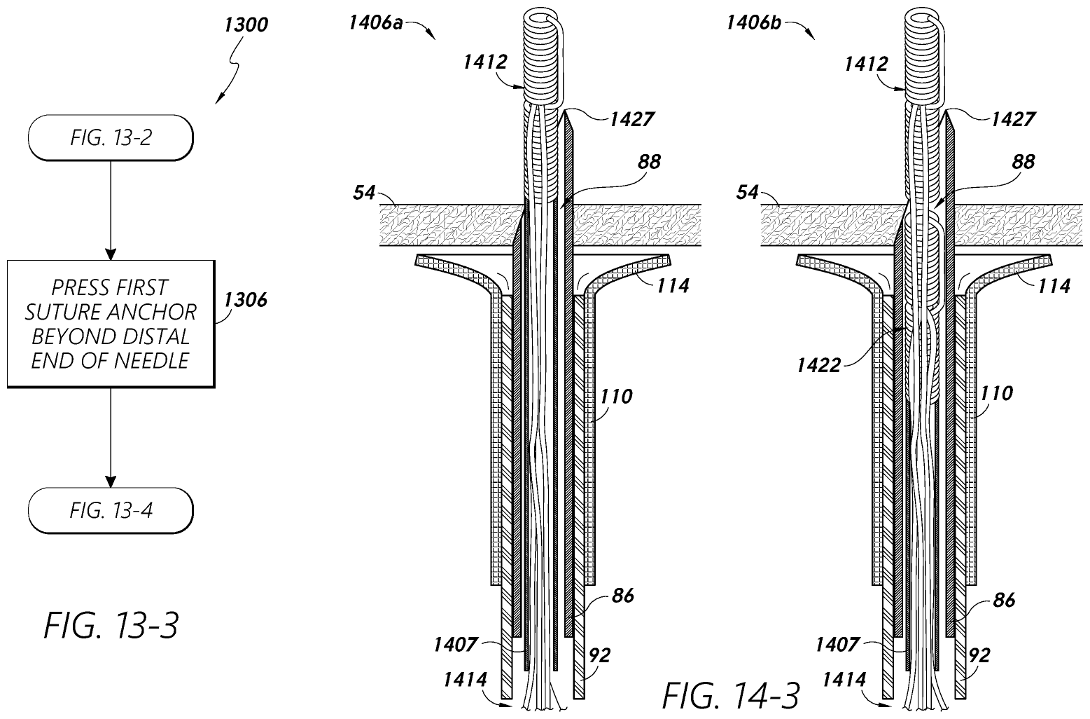

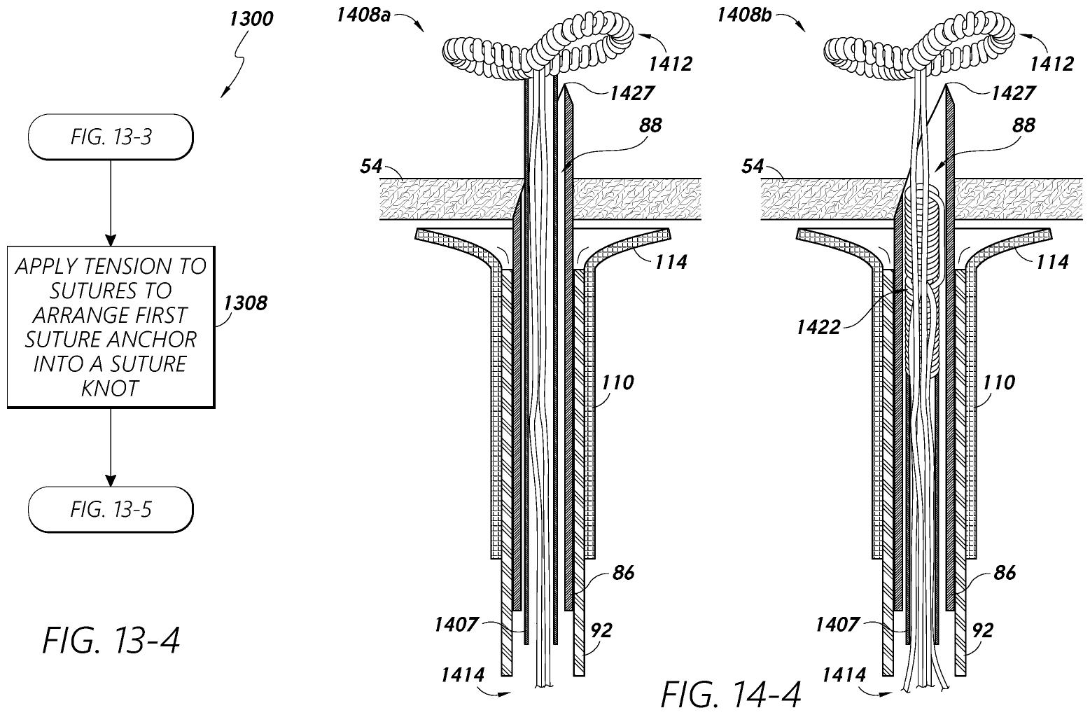

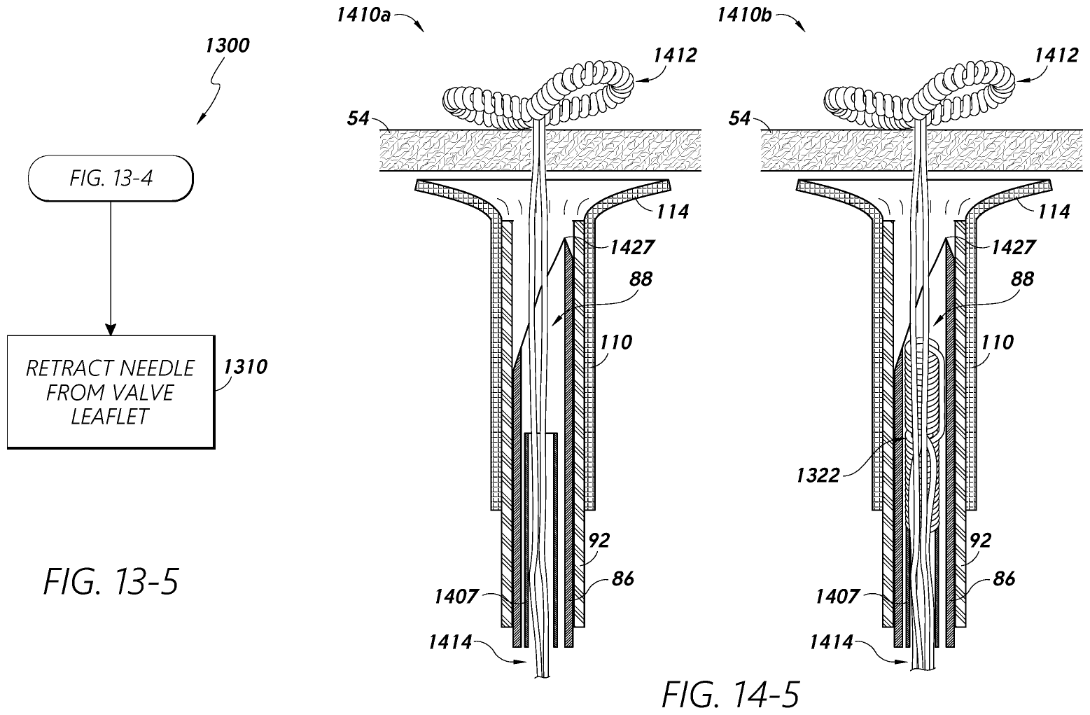

FIG. 13 (FIGS. 13-1, 13-2, 13-3, 13-4, and 13-5) is a flow diagram illustrating a process 1300 for delivering one or more leaflet anchors to a valve leaflet in accordance with one or more instances of the present disclosure. FIG. 14 (FIGS. 14-1, 14-2, 14-3, 14-4, and 14-5) provides illustrations corresponding to steps of the process 1300 described in FIG. 13.

At step 1302, the process 1300 involves delivering a needle 86 to a proximal side (e.g., underside) of a leaflet 54 of a valve (e.g., a mitral valve), as illustrated in images 1402a and 1402b of FIG. 14. The needle 86 may be delivered via one or more delivery devices, which can include a shaft no comprising an end effector 114. The needle 86 may be configured to pass through a lumen of the shaft no. Delivering the needle 86 to the leaflet 54 may involve contacting the end effector 114 with the proximal side of the leaflet 54. A pointed tip 1427 of the needle 86 may be positioned at or near the end effector 114 of the shaft no. The end effector 114 may advantageously be configured to contact the leaflet 54 without damaging the leaflet 54. In some instances, the needle 86 may be additionally and/or alternatively delivered via an outer tube 92. The outer tube 92 may comprise a lumen configured to receive the needle 86. Moreover, the outer tube 92 may be configured to fit at least partially within the lumen of the shaft no. While FIG. 14 illustrates truncated portions of the shaft no and/or tube 92, the shaft no and/or tube 92 may have any suitable length and/or may extend further than is shown in FIG. 14.

In some instances, the tip 1427 of the needle 86 may extend from an inner diameter of the needle 86. For example, the tip 1427 may be at least partially offset from the outer diameter of the needle 86 and/or from the inner diameter of the outer tube 92 while the needle 86 is within the outer tube 92.

During delivery of the needle 86 to the leaflet 54, a first suture anchor 1412 and/or second suture anchor 1422 may be situated at least partially within a lumen 88 of the needle 86. If only a single suture anchor is carried within the needle 86 (as shown in image 1402a), the first suture anchor 1412 may be pressed against a pusher. If multiple suture anchors are carried within the needle 86 (as shown in image 1402b), the first suture anchor 1412 and/or second suture anchor 1422 may be stacked end-to-end and/or in a single-file orientation within the needle 86 and/or may be pressed against a pusher 1407 extending through the lumen 88 of the needle 86. For example, the pusher 1407 may have an inner width and/or diameter that is less than a width and/or diameter of the first suture anchor 1412 and/or second suture anchor 1422. As a result, the first suture anchor 1412 and/or second suture anchor 1422 may not enter into a lumen of the pusher 1407 and/or may rest against a blunt edge of the pusher 1407. For example, in multi-anchor systems, the second suture anchor 1422 may rest against the blunt edge of the pusher 1407 and/or the first suture anchor 1412 may rest against the second suture anchor 1422. If more suture anchors are carried within the needle 86, additional suture anchors may be situated in an end-to-end and/or single-file orientation, similarly to the first suture anchor 1412 and/or second suture anchor 1422. Suture tails 1414 of the first suture anchor 1412 and/or second suture anchor 1422 may fit within and/or extend through the lumen of the pusher 1407.

The first suture anchor 1412 and/or second suture anchor 1422 may be wound into a coiled form while within the lumen 88 of the needle 86. For example, the first suture anchor 1412 and/or second suture anchor 1422 may be configured to assume a tubular and/or coiled form having a diameter approximately equal to an inner diameter of the needle 86. As a result, the needle 86 may be configured to prevent the first suture anchor 1412 and/or second suture anchor 1422 from unwinding while within the lumen 88 of the needle 86.

In some examples, the end effector 114 may be configured to contact the leaflet 54 without and/or substantially without damaging the leaflet 54. The end effector 114 may extend along the entire outer surface of the distal end of the shaft no such that only the end effector 114 of the shaft no may contact the leaflet 54.

At step 1304, the process 1300 involves puncturing the valve using the pointed tip 1427 of the needle 86, as shown in images 1404a and 1404b of FIG. 14. The needle 86 may be extended beyond the tube 92 and/or end effector 114 such that the pointed tip 1427 contacts the proximal side of the leaflet 54. The pointed tip 1427 may be configured to puncture the proximal side of the leaflet and/or one or more edges of the needle 86 may be configured to dilate the leaflet 54 without tearing and/or cutting the leaflet 54 after the puncture is made. The needle 86 may be extended at least partially beyond a distal side (e.g., upper side) of the leaflet 54. However, the needle 86 may only be extended as far as necessary to facilitate removal of the first suture anchor 1412 and/or second suture anchor 1422 beyond the distal side of the leaflet 54 to prevent damage to areas of anatomy at the distal side of the leaflet 54 by the pointed tip 1427.

At step 1306, the process 1300 involves extending the pusher 1407 to press the pusher 1407 against the first suture anchor 1412 and/or second suture anchor 1422 to cause the first suture anchor 1412 to exit the lumen 88 at the distal end of the needle 86 at the distal side of the leaflet 54, as shown in images 1406a and 1406b of FIG. 14. Where only a single suture anchor is within the needle 86 (as shown in image 1406a), the pusher 1407 may press against the first suture anchor 1412 to press the first suture anchor 1412 beyond the distal end of the needle 86. Where multiple suture anchors are within the needle 86 (as shown in image 1406b), the pusher 1407 may be pressed against the second suture anchor 1422, which may in turn press against the first suture anchor 1412 to cause the first suture anchor 1412 to exit the lumen 88 of the needle 86.

At step 1308, the process 1300 involves pulling on and/or otherwise applying tension to the suture tails 1414 of the first suture anchor 1412 to arrange and/or transform the first suture anchor 1412 into a bulky (e.g., FIG. 8) knot, as shown in images 1408a and 1408b of FIG. 14. In some instances, the suture tails 1414 may be accessible to a surgeon via the pusher 1407, needle 86, tube 92, and/or shaft no. The first suture anchor 1412 may be configured to twist and/or otherwise adjust while forming the bulky knot shape. The bulky knot shape may prevent the first suture anchor 1412 from retracting through the puncture opening in the leaflet 54. Where only one suture anchor is delivered via the needle 86 and/or is situated within the needle 86 (as shown image 1408a of FIG. 14), the pusher 1407 may be pressed against and/or may be in contact with the first suture anchor 1412 while tension is applied to the first suture anchor 1412.

At step 1310, the process 1300 involves retracting the needle 86 from the leaflet 54 while leaving the first suture anchor 1412 at the distal side of the leaflet 54, as shown in images 1410a and 1410b of FIG. 14. The suture tails 1414 of the first suture anchor 1412 may extend through the puncture opening of the leaflet 54 and/or may be secured to a ventricle wall using a pledget and/or other device(s).

Steps of the process 1300 may be repeated for additional suture anchors. For example, following deployment of the first suture anchor 1412, the needle 86 may be moved to another position of the leaflet 54 and/or to another leaflet to deploy the second suture anchor 1422. During deployment of the second suture anchor 1422, the pusher 1407 may be in contact with the second suture anchor 1422 as the second suture anchor 1422 is extended beyond the distal end of the needle 86 and/or while tension is applied to the second suture anchor 1422.

### Additional Examples

Described herein are various example medical sheaths and/or delivery methods. Some examples described herein may be used in combination and/or may be used independently.

Example 1: A delivery system comprising: a needle comprising a distal tip configured to pierce a valve leaflet of a heart and a lumen configured to receive one or more suture anchors; a first suture anchor configured to be delivered via the lumen of the needle; and a pusher configured to extend through the lumen of the needle and configured to press the first suture anchor beyond the distal tip of the needle.

Example 2: The delivery system of any example herein, in particular example 1, wherein the first suture anchor has a coiled and tubular form while within the lumen of the needle.

Example 3: The delivery system of any example herein, in particular example 1, further comprising a second suture anchor stacked end-to-end with the first suture anchor within the lumen of the needle.

Example 4: The delivery system of any example herein, in particular example 3, wherein the pusher is configured to contact the second suture anchor to press the first suture anchor beyond the distal tip of the needle.

Example 5: The delivery system of any example herein, in particular example 1, wherein suture tails of the first suture anchor are configured to extend through a lumen of the pusher.

Example 6: The delivery system of any example herein, in particular example 1, wherein the distal tip of the needle extends along an inner diameter of the needle.

Example 7: The delivery system of any example herein, in particular example 1, wherein the first suture anchor is configured to form a knot following removal from the needle.

Example 8: The delivery system of any example herein, in particular example 7, wherein the pusher is configured to press against the first suture anchor beyond the distal tip of the needle to facilitate forming the knot.

Example 9: A method comprising: delivering a needle carrying a first suture anchor within a lumen of the needle to a proximal side of a heart valve leaflet, the needle comprising a pointed tip at a distal end of the needle, the first suture anchor comprising a coiled suture and one or more suture tails; puncturing the proximal side of the heart valve leaflet with the pointed tip of the needle; extending the pointed tip beyond a distal side of the heart valve leaflet; pressing the first suture anchor beyond the distal end of the needle on the distal side of the heart valve leaflet; and tensioning the one or more suture tails of the first suture anchor to form the first suture anchor into a knot.

Example 10: The method of any example herein, in particular example 9, further comprising extending a pusher to press the first suture anchor out of the lumen of the needle.

Example 11: The method of any example herein, in particular example 10, further comprising maintaining contact between the pusher and the first suture anchor beyond the distal end of the needle.

Example 12: The method of any example herein, in particular example 11, further comprising maintaining contact between the pusher and the first suture anchor while tensioning the one or more suture tails of the first suture anchor.

Example 13: The method of any of any example herein, in particular example 10, wherein the pusher comprises a lumen configured to receive the one or more suture tails of the first suture anchor.

Example 14: The method of any of any example herein, in particular example 9, further comprising, before delivering the needle to the heart valve leaflet: winding the first suture anchor onto a suture winding tool; and placing the first suture anchor into the lumen of the needle at the distal end of the needle.

Example 15: The method of any example herein, in particular example 14, further comprising, before placing the first suture anchor in the lumen of the needle: winding a second suture anchor onto a suture winding tool; and placing the second suture anchor into the lumen of the needle at the distal end of the needle.

Example 16: The method of any of any example herein, in particular example 9, further comprising winding the first suture anchor into a tubular form on a suture winding tool.

Example 17: The method of any example herein, in particular example 16, further comprising placing the first suture anchor in the tubular form into the lumen of a needle at the distal end of the needle.

Example 18: The method of any example herein, in particular example 16, further comprising: winding a second suture anchor into a tubular form on the suture winding tool; and placing the second suture anchor into the lumen of the needle in an end-to-end configuration with the first suture anchor.

Example 19: The method of any of any example herein, in particular example 9, further comprising: extending a pusher through the lumen of the needle to press the first suture anchor beyond the distal end of the needle; and pressing the first suture anchor against the pusher to facilitate forming the knot.

Example 20: The method of any example herein, in particular example 19, wherein the pusher comprises a lumen configured to receive the one or more suture tails of the first suture anchor.

Depending on the example, certain acts, events, or functions of any of the processes or algorithms described herein can be performed in a different sequence, may be added, merged, or left out altogether. Thus, in certain instances, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain instances include, while other instances do not include, certain features, elements and/or steps. Thus, such conditional language is not generally intended to imply that features, elements and/or steps are in any way required for one or more instances or that one or more instances necessarily include logic for deciding, with or without author input or prompting, whether these features, elements and/or steps are included or are to be performed in any particular instance. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain instances require at least one of X, at least one of Y and at least one of Z to each be present.

It should be appreciated that in the above description of instances, various features are sometimes grouped together in a single instance, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular instance herein can be applied to or used with any other instance(s). Further, no component, feature, step, or group of components, features, or steps are necessary or indispensable for each instance. Thus, it is intended that the scope of the inventions herein disclosed and claimed below should not be limited by the particular instances described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example instances belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A delivery system comprising:
a needle comprising a distal tip configured to pierce a valve leaflet of a heart and a lumen configured to receive one or more suture anchors;
a first suture anchor configured to be delivered via the lumen of the needle; and
a pusher configured to extend through the lumen of the needle and configured to press the first suture anchor beyond the distal tip of the needle.

2. The delivery system of claim 1, wherein the first suture anchor has a coiled and tubular form while within the lumen of the needle.

3. The delivery system of claim 1, further comprising a second suture anchor stacked end-to-end with the first suture anchor within the lumen of the needle.

4. The delivery system of claim 3, wherein the pusher is configured to contact the second suture anchor to press the first suture anchor beyond the distal tip of the needle.

5. The delivery system of claim 1, wherein suture tails of the first suture anchor are configured to extend through a lumen of the pusher.

6. The delivery system of claim 1, wherein the distal tip of the needle extends along an inner diameter of the needle.

7. The delivery system of claim 1, wherein the first suture anchor is configured to form a knot following removal from the needle.

8. The delivery system of claim 7, wherein the pusher is configured to press against the first suture anchor beyond the distal tip of the needle to facilitate forming the knot.

9. A method comprising:
delivering a needle carrying a first suture anchor within a lumen of the needle to a proximal side of a heart valve leaflet, the needle comprising a pointed tip at a distal end of the needle, the first suture anchor comprising a coiled suture and one or more suture tails;
puncturing the proximal side of the heart valve leaflet with the pointed tip of the needle;
extending the pointed tip beyond a distal side of the heart valve leaflet;
pressing the first suture anchor beyond the distal end of the needle on the distal side of the heart valve leaflet; and
tensioning the one or more suture tails of the first suture anchor to form the first suture anchor into a knot.

10. The method of claim 9, further comprising extending a pusher to press the first suture anchor out of the lumen of the needle.

11. The method of claim 10, further comprising maintaining contact between the pusher and the first suture anchor beyond the distal end of the needle.

12. The method of claim 11, further comprising maintaining contact between the pusher and the first suture anchor while tensioning the one or more suture tails of the first suture anchor.

13. The method of claim 10, wherein the pusher comprises a lumen configured to receive the one or more suture tails of the first suture anchor.

14. The method of claim 9, further comprising, before delivering the needle to the heart valve leaflet:
winding the first suture anchor onto a suture winding tool; and
placing the first suture anchor into the lumen of the needle at the distal end of the needle.

15. The method of claim 14, further comprising, before placing the first suture anchor in the lumen of the needle:
winding a second suture anchor onto a suture winding tool; and
placing the second suture anchor into the lumen of the needle at the distal end of the needle.

16. The method of claim 9, further comprising winding the first suture anchor into a tubular form on a suture winding tool.

17. The method of claim 16, further comprising placing the first suture anchor in the tubular form into the lumen of a needle at the distal end of the needle.

18. The method of claim 16, further comprising:
winding a second suture anchor into a tubular form on the suture winding tool; and
placing the second suture anchor into the lumen of the needle in an end-to-end configuration with the first suture anchor.

19. The method of claim 9, further comprising:
extending a pusher through the lumen of the needle to press the first suture anchor beyond the distal end of the needle; and
pressing the first suture anchor against the pusher to facilitate forming the knot.

20. The method of claim 19, wherein the pusher comprises a lumen configured to receive the one or more suture tails of the first suture anchor.
